(19) United States
(12) Patent Application Publication
(10) Pub. No.: US 2025/0354931 A1
(43) Pub. Date: Nov. 20, 2025

(54) SPATIAL TARGETING OF ANALYTES

(71) Applicant: 10x Genomics, Inc., Pleasanton, CA (US)

(72) Inventors: Preyas Shah, Pleasanton, CA (US); Eswar Prasad Ramachandran Iyer, Sunnyvale, CA (US)

(21) Appl. No.: 19/291,081

(22) Filed: Aug. 5, 2025

Related U.S. Application Data
(62) Division of application No. 17/172,816, filed on Feb. 10, 2021, now Pat. No. 12,399,123.
(60) Provisional application No. 62/976,783, filed on Feb. 14, 2020.

(51) Int. Cl. G01N 21/64; G01N 1/02; G01N 1/06; G01N 1/28; G01N 33/543; G02B 21/00; G02B 21/06

(52) U.S. Cl. CPC G01N 21/6452; G01N 21/6456; G01N 33/543; G02B 21/0016; G02B 21/06; G01N 2001/028; G01N 2001/068; G01N 2001/284; G01N 2021/6419; G01N 2021/6421

(57) ABSTRACT

Systems and methods are provided for targeting one or more features in a region of interest, and efficiently removing the features from an array of features. The systems and methods can remove one or more beads that contain analytes, intermediate agents, or a combination thereof, from the region of interest of a substrate by emitting light toward the beads on the substrate.

### CROSS-REFERENCE TO RELATED APPLICATION

This application is a divisional of U.S. patent application Ser. No. 17/172,816, filed Feb. 10, 2021, which claims the benefit of U.S. Patent Application Ser. No. 62/976,783 titled SPATIAL TARGETING OF ANALYTES, filed Feb. 14, 2020, the disclosure of which is hereby incorporated by reference in its entirety.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Users may be interested in focusing on particular regions of interest of a biological sample. Techniques are needed to identify a region of interest and collect analytes from the region of interest in an efficient and reliable manner.

### SUMMARY

This document generally relates to apparatuses, systems, and methods for preparing a sample, such as a biological sample. In particular, the present disclosure provide systems and methods for spatially targeting one or more analytes in a region of interest, and efficiently obtaining the analytes from a biological sample.

Some implementations of the present disclosure provide systems and methods for removing one or more features (e.g., beads) that contain analytes of interest from a substrate by emitting light toward the features on the substrate. In some implementations, a feature capture device is provided. The feature capture device can include a capture surface configured to face at least the features of interest on the substrate. The capture surface can be activated to attach (e.g., adhesively attach) the features of interest when a beam of light is projected on an area of the capture surface that corresponds to the features of interest on the substrate. For example, the feature capture device can include a transparent body having a first surface and a second surface that, for example, may be arranged opposite to the first surface. A light beam can enter the first surface of the feature capture device, pass through the transparent body, and reach the second surface that includes the capture surface. In some implementations, a light source can emit a beam of light with a predetermined beam divergence that is suitable for a particular size of region of interest. For example, light can be a laser of a particular wavelength configured to activate a limited region on the substrate.

Some implementations of the present disclosure can include an instrument for identifying a region of interest on an array of features affixed to the substrate. Examples of such an instrument include a microscope, a camera, or other suitable imaging devices configured to visualize the array of features on the substrate. A region of interest in the array of features can be manually determined by a user who examines the array through the instrument. Alternatively or in addition, the instrument can be configured to automatically identify a region of interest in the array of features based on one or more inputs or factors, such as particular types of analytes of interest (e.g., cell type, RNA, Protein, DNA, etc.), particular research purposes, etc.

Once the feature capture device has captured the features of interest, it can be removed from the position where it captured the features from the substrate, and processed to retrieve the captured features from the capture surface of the feature capture device. Various methods can be used to retrieve the captured features from the feature capture device.

In alternative implementations, the present disclosure provides a substrate that attaches an array of features with photo-sensitive bonds, such as cleavable linkers. A region of interest can be identified on the feature array using, for example, the instrument described herein, and a beam of light is projected onto the region of interest so that the photo-sensitive bonds affixing the features in the region of interest are cleaved, and the features in the region of interest are released from the substrate. For example, a light beam can be projected onto a bottom surface of the substrate that is opposite to a top surface on which the feature array is affixed with the photo-sensitive bonds. The light beam passes through the transparent body of the substrate and reaches the top surface to cleave the photo-sensitive bonds. In some implementations, a light source emits a beam of light with a predetermined beam divergence that is suitable for a particular size of region of interest. For example, light can be a laser of a particular wavelength (e.g., ultraviolet (UV) light) configured to be projected on a limited region on the substrate. In some implementations, the removed features can be collected within a reservoir containing a suitable liquid.

Particular embodiments described herein include a system for preparing a biological sample. The system includes a substrate, a feature capture block, a light generator, and a controller. The substrate is configured to place a feature array thereon. The array of features may be configured to capture analytes from the biological sample. The feature capture block may include a capture surface configured to face the feature array on the substrate. The light generator may be configured to emit light. The controller may be configured to activate the light generator to emit light toward the capture surface of the feature capture block to permit the capture surface to capture at least one feature from the feature array.

In some implementations, the system can optionally include one or more of the following features. The feature capture block may include a transparent body to permit light to pass through the body. The transparent body of the feature capture block may have a light entering surface onto which the light is emitted. The light may pass through the transparent body and be directed toward the capture surface. The light entering surface may be arranged to be opposite to the capture surface. The capture surface may include a feature capture material. The feature capture material may include a light-activated or heat-activated adhesive. The light-activated adhesive may include a polymer which can be triggered to be adhesive under selected conditions. The feature capture block may be configured to be arranged such that the capture surface contacts with the feature array on the substrate. The feature capture block may be configured to be arranged such that the capture surface is spaced apart from the feature array on the substrate. The light generator may be configured to generate a laser of a predetermined wavelength. The laser may include an ultraviolet (UV) laser. Other light sources can also be used, such as a Digital Micromirror Device (DMD). The system may include a microscope configured to visualize a region of interest on the feature array. The controller may be configured to activate the light generator to emit the light toward a portion of the capture surface corresponding to the region of interest on the feature array. The light can be applied to the portion of the capture surface directly from the light generator. Alternatively, the light can be deflected from the light generator and then interact with the portion of the capture surface. The light-emitted portion of the capture surface may be permitted to capture at least one feature in the region of interest on the feature array. The feature array may include a spatially-barcoded bead array. The feature array may include a plurality of hydrogel beads. Alternatively or in addition, the feature array may include a plurality of silica beads, which may have an average diameter of 50 microns or smaller (e.g., nanometer scale). The feature array may include a plurality of beads being uniform in size (e.g., diameter). The feature array may be affixed to the substrate in a monolayer.

Particular embodiments described herein include a method of preparing a biological sample. The method may include providing a substrate affixing a feature array thereon, the feature array capturing analytes from the biological sample; arranging a feature capture block relative to the substrate such that a capture surface of the feature capture block faces the feature array on the substrate; identifying a region of interest on the feature array; emitting light (either directly or after deflected) toward a portion of the capture surface of the feature capture block, the portion of the capture surface corresponding to the region of interest on the feature array; permitting for the light-emitted portion of the capture surface to capture at least one feature within the region of interest on the feature array; removing the feature capture block from the substrate; and retrieving the at least one feature from the capture surface of the feature capture block.

In some implementations, the method can optionally include one or more of the following attributes. The method may include placing the substrate on a substrate stage of a microscope to visualize the feature array on the substrate including the region of interest. The feature capture block may include a transparent body to permit the light to pass through the body. The transparent body of the feature capture block may have a light entering surface onto which the light is emitted. The light may pass through the transparent body and be directed toward the capture surface. The light entering surface may be arranged to be opposite to the capture surface. The capture surface may include a feature capture material. The feature capture material may include a light-or heat-activated adhesive. The light-activated adhesive may include a polymer which can be triggered to be adhesive under selected conditions. The feature capture block may be configured to be arranged such that the capture surface contacts with the feature array on the substrate. The feature capture block may be configured to be arranged such that the capture surface is spaced apart from the feature array on the substrate. The light may include a laser of a predetermined wavelength. The light may include an ultraviolet (UV) laser. Other light sources can also be used, such as a Digital Micromirror Device (DMD). The feature array may include a spatially-barcoded bead array. The feature array may include a plurality of beads being uniform in size (e.g., diameter) and arranged in a monolayer.

Particular embodiments described herein include a system for preparing a biological sample. The system may include a substrate, a light generator, and a controller. The substrate may be configured to include photo-sensitive bonds and a feature array attached to the substrate via the photo-sensitive bonds. The feature array may be configured to capture analytes from the biological sample. The light generator may be configured to emit light. The controller may be configured to activate the light generator to emit the light toward a region of interest of the feature array to cleave the photo-sensitive bonds in the region of interest and permit for at least one feature within the region of interest of the feature array to be released from the substrate.

In some implementations, the system can optionally include one or more of the following features. The substrate may include a transparent body having a first surface and a second surface. The first surface may be configured to include the photo-sensitive bonds and the feature array. The light generator may be configured to emit the light onto the second surface of the substrate such that the light passes through the transparent body and reaches the photo-sensitive bonds on the first surface of the substrate. The first surface may be arranged to be opposite to the second surface. The light generator may be configured to generate a laser of a predetermined wavelength. The laser may include an ultraviolet (UV) laser. Other light sources can also be used, such as a Digital Micromirror Device (DMD). The system may include a reservoir containing a fluid into which the at least one feature is released from the substrate. The photo-sensitive bonds may include photo-cleavable linkers. The photo-cleavable linkers may include at least one of photo-sensitive chemical bonds include -amino-3-(2-nitrophenyl) propionic acid (ANP), phenacyl ester derivatives, 8-quinolinyl benzenesulfonate, dicoumarin, 6-bromo-7-alkixycoumarin-4-ylmethoxycarbonyl, a bimane-based linker, or a bis-arylhydrazone based linker. The system may include a microscope configured to visualize a region of interest on the feature array. The controller may be configured to activate the light generator to emit the light toward a portion of the substrate corresponding to the region of interest on the feature array. The feature array may include a spatially-barcoded bead array. The feature array may include a plurality of hydrogel beads. The feature array may include a plurality of beads being uniform in size (e.g., diameter). The feature array may be affixed to the substrate in a monolayer.

Particular embodiments described herein include a method of preparing a biological sample. The method may include providing a substrate affixing a feature array using photo-sensitive bonds; identifying a region of interest on the feature array; emitting light toward a portion of the substrate, the portion of the capture surface corresponding to the region of interest on the feature array; permitting for the photo-sensitive bonds in the region of interest to be cleaved so that at least one feature within the region of interest is released from the substrate; and collecting the at least one feature.

In some implementations, the method can optionally include one or more of the following features. The method may include placing the substrate on a substrate stage of a microscope to visualize the feature array on the substrate including the region of interest. The substrate may include a transparent body having a first surface and a second surface. The first surface configured to place the photo-sensitive bonds and the feature array. Emitting light may include emitting light onto second surface of the substrate such that the light passes through the transparent body and reaches the photo-sensitive bonds on the first surface of the substrate. The first surface may be arranged to be opposite to the second surface. The light may include a laser of a predetermined wavelength. The light may include an ultraviolet (UV) laser. Other light sources can also be used, such as a Digital Micromirror Device (DMD). Collecting the at least one feature may include collecting the at least one feature within a fluid contained in a reservoir. The photo-sensitive bonds may include photo-cleavable linkers. The photo-cleavable linkers may include at least one of photo-sensitive chemical bonds include 3-amino-3-(2-nitrophenyl)propionic acid (ANP), phenacyl ester derivatives, 8-quinolinyl benzenesulfonate, dicoumarin, 6-bromo-7-alkixycoumarin-4-ylmethoxycarbonyl, a bimane-based linker, or a bis-arylhydrazone based linker. The feature array may include a spatially-barcoded bead array. The feature array may include a plurality of hydrogel beads. The feature array may include a plurality of beads being uniform in size (e.g., diameter). The feature array may be affixed to the substrate in a monolayer.

As such, the present disclosure provides solutions for efficiently performing spatially resolved capture of analytes by physically dissecting a region of interest from captured analytes on the feature array.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 illustrates an exemplary system for preparing a biological sample.

FIGS. 2A-C illustrate an exemplary process for performing spatially-targeted feature capture.

FIG. 3 is a flowchart of an exemplary process for performing spatially-targeted feature capture.

FIG. 4 illustrates another exemplary system for preparing a biological sample.

FIGS. 5A-C illustrate an exemplary process for performing spatially-targeted feature capture.

FIG. 6 is a flowchart of an exemplary process for performing spatially-targeted feature capture.

FIG. 7 is a block diagram of computing devices that may be used to implement the systems and methods described in this document, as either a client or as a server or plurality of servers.

### DETAILED DESCRIPTION

In general, the present disclosure relates to devices, instruments, systems, and methods for preparing a biological sample. In particular, the present disclosure provide systems and methods for spatially targeting one or more analytes in a region of interest, and efficiently removing the analytes from a biological sample. Some implementations of the present disclosure provide systems and methods for removing one or more beads that contain analytes of interest or intermediate agents representative of analytes of interest (e.g., an analyte capture agent or a ligation product) from a substrate by emitting light toward the beads on the substrate.

In some implementations, the present disclosure provides a bead capture block that includes a capture surface configured to face at least the beads of interest on the substrate. The capture surface can be activated to adhere to the beads of interest when a beam of light is projected on an area of the capture surface that corresponds to the beads of interest on the substrate. For example, the bead capture block can include a transparent body having a first surface and a second surface that, for example, may be arranged opposite to the first surface. A light beam can enter the first surface of the bead capture block, pass through the transparent body, and reach the second surface that includes the capture surface. Various types of light sources can be selected depending on various factors, such as the characteristics of the capture surface. In some implementations, a light source is configured to emit a beam of light with a predetermined beam divergence that is suitable for a particular size of region of interest. For example, light can be a laser of a particular wavelength configured to activate a limited region on the substrate. Examples of the wave length include 250-350 nm or 350-460 nm UV light.

Some implementations of the present disclosure can include an instrument for identifying a region of interest on an array of beads affixed to the substrate. Examples of such an instrument include a microscope, a camera, or other suitable imaging devices configured to visualize the array of beads on the substrate. A region of interest in the bead array can be manually determined by a user who examines the bead array through the instrument. Alternatively or in addition, the instrument is configured to automatically identify a region of interest in the bead array based on one or more inputs or factors, such as particular types of analytes of interest (e.g., cell type such as immune cells or cancer cells), particular research purposes (e.g., diagnosis), etc.

Once the bead capture block has captured the beads of interest, it can be removed from the position that it captured the beads from the substrate, and processed to retrieve the captured beads from the capture surface of the bead capture block. Various methods can be used to retrieve the captured beads from the bead capture block.

In alternative implementations, the present disclosure provides a substrate that attaches an array of beads with photo-sensitive bonds, such as cleavable linkers. A region of interest can be identified on the bead array using, for example, the instrument described herein, and a beam of light is projected onto the region of interest so that the photo-sensitive bonds affixing the beads in the region of interest are cleaved, and the beads in the region of interest are released from the substrate. For example, a light beam can be projected onto a bottom surface of the substrate that is opposite to a top surface on which the bead array is affixed with the photo-sensitive bonds. The light beam passes through transparent body of the substrate and reaches the top surface to cleave the photo-sensitive bonds. Various types of light sources can be selected based on various factors, such as the characteristics of the substrate and the photo-sensitive bonds being used. In some implementations, a light source is configured to emit a beam of light with a predetermined beam divergence that is suitable for a particular size of region of interest. For example, light can be a laser of a particular wavelength (e.g., ultraviolet (UV) light) configured to activate a limited region on the substrate. In some implementations, the removed beads can be collected within a reservoir (e.g., containing a suitable liquid). In some implementations, the released beads can be attached to an additional substrate (e.g., a flow cell, a wafer, a bead, a slide) using a covalent bond, such as through photo-click chemistry. For example, the released beads and the additional substrate can include complementary reactive moieties (e.g., a terminal alkene and a tetrazine or a tetrazole) that can perform a cycloaddition reaction upon irradiation with an appropriate wavelength of light, thereby covalently linking the released beads to the additional substrate. Additional examples of photo-click chemistry are described in Oliveira, et al. Chemical Society Reviews 46.16 (2017): 4895-4950, incorporated herein by reference.

The techniques described herein allow a user to obtain analytes in a targeted area for analysis before and after a biological sample is thoroughly analyzed. Further, the substrate that captures an array of features that capture analytes can be used for analysis of a particular region of interest, and can be used again for analysis of another region of interest simultaneously or at a later time. In some implementations, the feature array can include spatially-barcoded capture probes so that the analytes included in the target features removed from a targeted area can be accurately identified in a spatial analysis, as described herein.

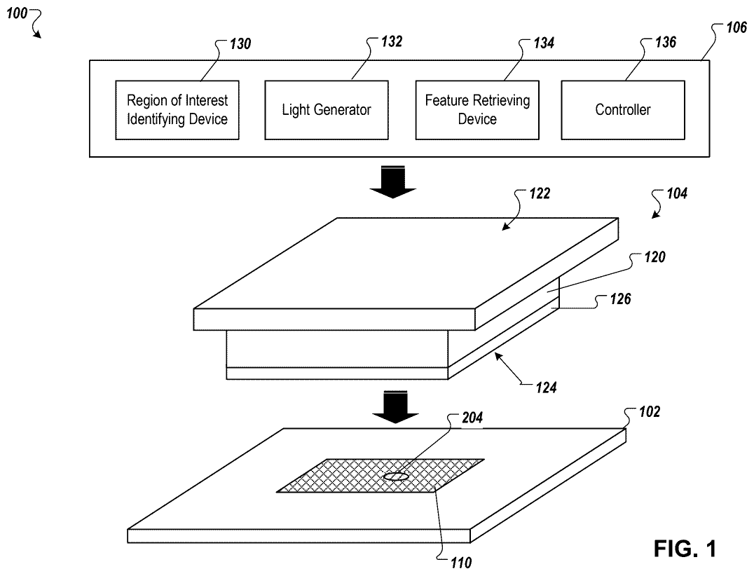

Referring to FIGS. 1-3, an example system for preparing a biological sample is described. In some implementations, the system is configured to obtain one or more analytes that are spatially targeted. For example, the system can be configured to remove one or more features that contain analytes of interest from a substrate by projecting light toward the features of interest on the substrate.

FIG. 1 illustrates an exemplary system 100 for preparing a biological sample. The system 100 includes a substrate 102, a targeted feature capture device 104, and a targeted sample preparation system 106.

In some implementations, the substrate 102 is configured to position an array of features 110. The feature array 110 can include spatially-barcoded capture probes, or an array of beads with such capture probes. The array of features 110 can be directly or indirectly attached or fixed to the substrate 102, or disposed on the substrate 102 in various manners described herein. The array of features 110 can include spatially-barcoded features for array-based spatial analysis, as described herein.

The features in the feature array 110 can include analytes captured from a biological sample, such as a cell or a tissue. Therefore, the substrate 102 does not include the biological sample (e.g., cell, tissue, etc.), but includes the feature array. Alternatively, the features in the feature array 110 can include intermediate agents (e.g., analyte capture agents and/or ligation products) obtained after interacting with analytes in the biological sample. Therefore, the substrate 102 does not include the biological sample (e.g., cell, tissue, etc.), but includes the feature array. Various systems and methods can be used to capture analytes from a sample using the feature array 110. For example, the substrate 102 with the feature array 110 can be used for array-based spatial analysis, as described herein. For example, an array-based spatial analysis method described herein can be used to transfer one or more analytes from a sample to the feature array 110 on the substrate. Each feature may be associated with a unique spatial location on the array, and used to identify the analytes and the spatial location of each analyte based on the relative spatial location of the feature to which that the analyte is bound on the feature array 110. Exemplary array-based spatial analysis methods are further described herein.

The targeted feature capture device 104 is configured to selectively retrieve one or more of the features that are of interest from the feature array 110. The targeted feature capture device 104 is configured to be positioned at a distance from the feature array 110 of the substrate 102, or abutted to the feature array 110 of the substrate 102 so that one or more features of interest can be captured by the targeted feature capture device 104.

In some implementations, the feature capture device 104 includes a body 120 having a light receiving surface 122 and a capture surface 124. The body 120 can be configured as a block that is transparent so that light can pass through. The body 120 can be made of various materials, such as polymer, glass, and other suitable material having desired light transmittance. As described herein, the light receiving surface 122 of the body 120 provides a surface that receives light emitted thereon. The capture surface 124 of the body 120 is configured to be disposed relative to the feature array 110 on the substrate 102. For example, the capture surface 124 of the body 120 can be positioned at a distance from the feature array 110 on the substrate 102. Alternatively, the capture surface 124 can be positioned to be abutted to the feature array 110 of the substrate 102.

The capture surface 124 is configured to capture one or more features from the feature array 110 on the substrate 102 when the light is projected on a region of the capture surface 124 that corresponds to the features in the feature array 110. In some implementations, the capture surface 124 includes a selective adhesive layer 126 that can be triggered to be adhesive under predetermined conditions. For example, the selective adhesive layer 126 is not adhesive under normal conditions (e.g., when not activated by light or heat), and becomes adhesive only when one or more predetermined conditions (e.g., when activated by light of a certain wavelength or heat of a certain temperature) are satisfied. In some implementations, the selective adhesive layer 126 includes a light-activated adhesive material that is activated to be adhesive when light of predetermined characteristic is projected onto the layer 126. Examples of such a light-activated adhesive material include special polymer and other suitable materials. The light-activated adhesive material can be provided to the capture surface 124 in various manners. In one example, the light-activated adhesive material can be coated on the capture surface 124 of the body 120.

The targeted sample preparation system 106 is configured to identify a region of interest 204 on the feature array 110 and retrieve selected features from the feature array 110 using the targeted feature capture device 104. In some implementations, the targeted sample preparation system 106 can include a region of interest (ROI) identifying device 130, a light generator 132, a feature retrieving device 134, and a controller 136.

The ROI identifying device 130 is configured to identify a ROI 204 on the feature array 110 so that one or more features are selectively retrieved from the feature array 110. In some implementations, the ROI identifying device 130 can take an image of the feature array 110 on the substrate 102 in real-time and provide the image to a user so that the user can visualize the feature array 110. For example, the ROI identifying device 130 can include, or be connected to, a display device (e.g., a display screen), and stream the image of the feature array 110 to the display device that can output the image. Examples of the ROI identifying device 130 can include a microscope, a camera, or other suitable imaging devices configured to visualize the feature array 110 of the substrate 102.

In some implementations, the ROI identifying device 130 is configured to automatically identify a ROI on the feature array 110 based on one or more inputs or factors, such as particular types of analytes of interest, particular research purposes, etc. For example, the ROI identifying device 130 can employ a machine learning model that is trained for one or more analysis or research purposes, and configured to automatically determine a ROI on the feature array 110 to meet a particular analysis or research purpose.

In some implementations, a biological sample can be stained to facilitate visualization. Example methods for staining a sample are described herein. The ROI identifying device 130 can capture an image of the stained sample. Optionally, the ROI identifying device 130 can interpret low pass sequencing data (e.g., via amplification of second strand synthesis on the sample removed from the substrate). The removed sample can be placed back on the substrate. The ROI identifying device 130 can visually identify a region of interest on the image based on the stain and/or the sequencing data that have been optionally obtained. The ROI identifying device 130 can be used to permit a user to select a ROI based on the stain image. In some implementation, the ROI identifying device 130, which may employ a machine learning algorithm, can automatically identify and mark a ROI on the stained image. In some implementations, the user can control the light generator 132 to emit light toward the identified ROI on the feature array 110 through the feature capture device 104. In other implementations, the light generator 132 can be automatically controlled and guided to be placed so that light is emitted toward the identified ROI on the feature array 110 through the feature capture device 104.

The light generator 132 can be configured to generate and emit light on the substrate 102 through the feature capture device 104. In some implementations, the light generator 132 is manually controllable to be positioned relative to the substrate 102 and/or the feature capture device 104, and also manually controllable to generate and emit light to a particular area (e.g., the identified ROI) on the feature array 110 of the substrate 102. For example, the light generator 132 can include a user interface (e.g., physical or virtual buttons, switches, or other suitable input devices) for receiving user input of controlling the light generator 132. Alternatively or in addition, the light generator 132 is configured to automatically adjust its position relative to the substrate 102 and/or the feature capture device 104 and generate and emit light toward a particular area (e.g., the identified ROI) on the feature array 110 of the substrate 102.

The feature retrieving device 134 is configured to retrieve features from the feature capture device 104. For example, when the features are captured at the capture surface 124 of the feature capture device 104, the feature retrieving device 134 is used to release the features from the capture surface 124 of the feature capture device 104. The feature retrieving device 134 can use various configurations and methods to retrieve the features from the feature capture device 104. For example, the feature retrieving device 134 can provide a dissolvent to dissolve a material (e.g., the selective adhesive layer 126) coated on the capture surface 124 so that the features are released from the feature capture device 104.

The controller 136 can be configured to control the ROI identifying device 130, the light generator 132, and/or the feature retrieving device 134. In some implementations, the controller 136 is configured to control the ROI identifying device 130 to identify the ROI on the feature array 110 and receive data indicative of the identified ROI. The controller 136 can control the light generator 132 to arrange or orient the light generator 132 in a desired position relative to the feature array 110 on the substrate 102, and/or generate and project light onto the identified ROI on the feature array 110 through the feature capture device 104 so that the features on the identified ROI are captured to the capture surface 124 of the feature capture device 104. The controller 136 can further control the feature retrieving device 134 to release the captured features from the capture surface 124 for further analysis.

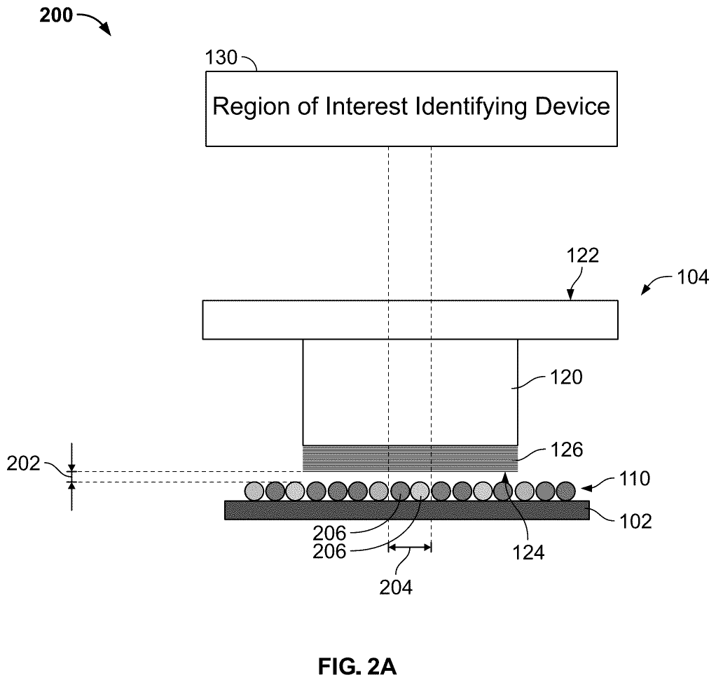

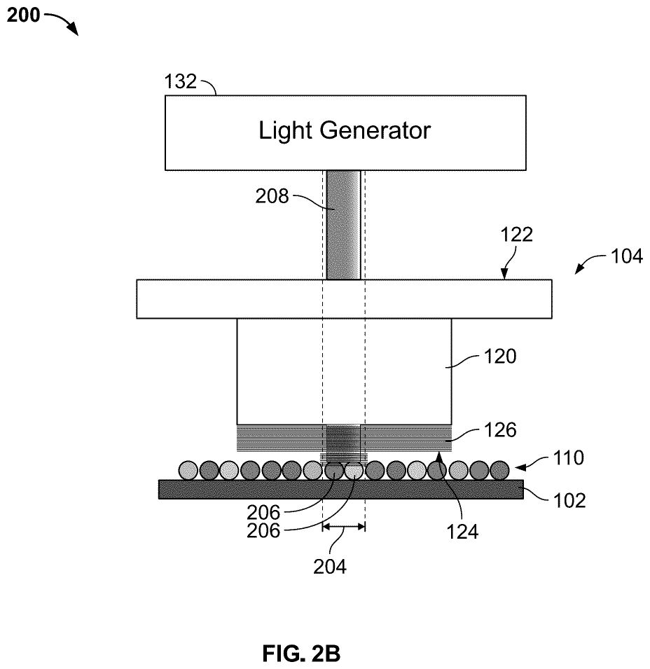

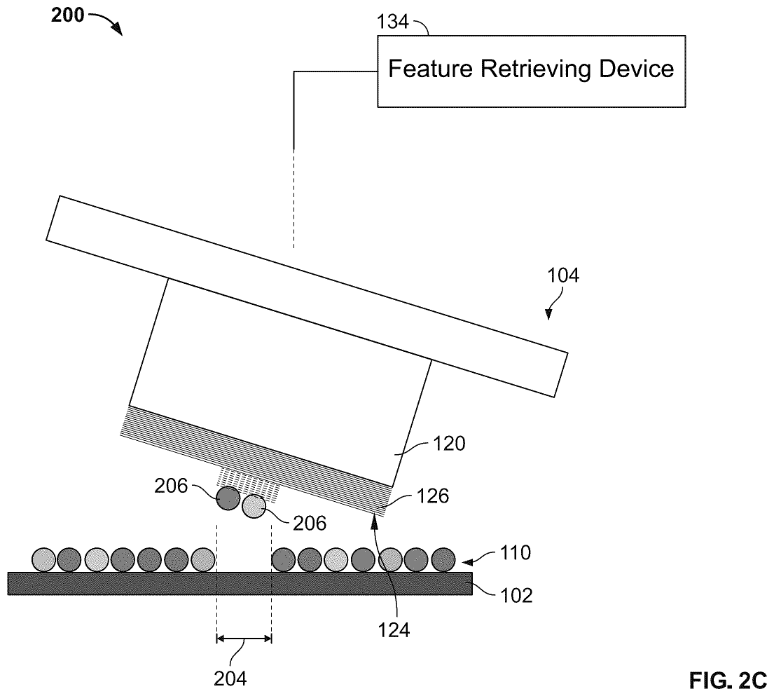

FIGS. 2A-C illustrate an exemplary process 200 for performing spatially-targeted feature capture. In this example, the process 200 is illustrated to be performed using the system 100 described in FIG. 1. Referring to FIG. 2A, the process 200 can be performed after the feature array 110 on the substrate 102 has captured analytes from a biological sample. The feature capture device 104 is arranged to a desired position relative to the substrate 102. For example, the feature capture device 104 is arranged such that the capture surface 124 faces at least a portion of the feature array 110 on the substrate 102. In some implementations, the feature capture device 104 can be arranged such that the capture surface 124 is spaced apart at a distance 202 from the feature array 110 on the substrate 102. The distance 202 can be determined to be small enough for the material 126 at the capture surface 124 to extend toward and adhere to targeted features of the feature array 110, when the material 126 is triggered by a projected light as described herein. The distance 202 can range between 0 to 100 microns in some examples. In another example, the distance 202 can range between 0 to 50 microns. A bigger distance 202 is possible depending on, for example, the characteristics (e.g., a degree of stretching when light-activated) of the material 126 at the capture surface 124. In other implementations, the feature capture device 104 can be arranged such that the capture surface 124 contacts with at least a portion of the feature array 110.

In some implementations, the ROI identifying device 130 can be used to identify a region of interest (ROI) 204 in the feature array 110 on the substrate 102. The ROI 204 is determined to include one or more target features 206 among the feature array 110. As described herein, the ROI identifying device 130 can provide a visual image of the feature array 110 to a user so that the user can identify the ROI 204 from the visual image of the feature array 110. In addition or alternatively, the ROI identifying device 130 can automatically identify the ROI 204, and mark the ROI 204 on the image of the feature array 110, so that light can be accurately projected onto the ROI 204.

Referring to FIG. 2B, the light generator 132 is used to emit light 208 onto the ROI 204 of the feature array 110. In some implementations, the light generator 132 generates a beam of light 208 of suitable wavelength and projects the light beam 208 onto the ROI 204. For example, the light beam 208 can be a laser of suitable wavelength.

In some implementations, the light 208 is projected onto the light receiving surface 122, and passes through the transparent body 120 to reach the capture surface 124. For example, the light receiving surface 122 is arranged opposite to the capture surface 124 so that the light 208 travels a linear path through the body 120. Alternatively, the light receiving surface 122 can be arranged a different side of the body 120 so that the light 208 is projected onto such a different side of the body 120 and travels along a non-linear path from the light receiving surface 122 to the capture surface 124. In this configuration, one or more reflectors can be included in the body 120 to change the path of the light 208, or the interior of the body 120 can be configured to form a non-linear light path toward the capture surface 124.

The light 208 can locally activate the layer 126 at the capture surface 124 so that the layer 126 becomes adhesive at the area (e.g., the ROI 204) that receives the light 208. For example, the layer 126 can absorb the energy from the light 208 and change its properties to become adhesive. The area of the layer 126 that becomes locally adhesive can attach to the light-targeted features 206 on the substrate 102.

Referring to FIG. 2C, the feature capture device 104 that attaches the light-targeted features 206 can be removed from the substrate 102. The light-targeted features 206 can be retrieved from the capture surface 124 of the feature capture device 104.

In some implementations, the feature retrieving device 134 can be used to release the light-targeted features 206 from the capture surface 124. As described herein, for example, the feature retrieving device 134 can include a chamber that contains a dissolvent for dissolving a material (e.g., the selective adhesive layer 126) coated on the capture surface 124. The feature retrieving device 134 can be at least partially immersed in the chamber so that the features are released from the feature capture device 104. Alternatively, the feature retrieving device 134 can provide a spray or other types of injectors for discharging a dissolvent toward the material (e.g., the selective adhesive layer 126) coated on the capture surface 124 so that the features are released from the feature capture device 104.

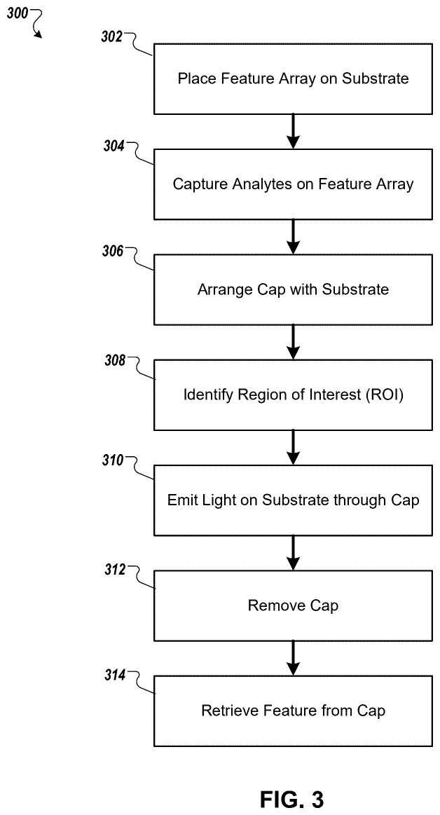

FIG. 3 is a flowchart of an example process 300 for performing spatially-targeted feature capture. In some implementations, the process 300 can be at least partially performed using the system 100 described in FIG. 1. In addition or alternatively, the process 300 can be used to implement at least part of the process 200 described in FIGS. 2A-C.

The process 300 can include placing a feature array on a substrate (302). The feature array can include spatially-barcoded capture probes, or an array of beads with such capture probes or functionality. For example, the feature array can be directly or indirectly attached or fixed to the substrate. Other methods can be used to place the feature array on the substrate.

The process 300 can include capturing analytes from a biological sample on the feature array of the substrate (304). Various methods can be used for capturing analytes from a sample. In one example, a biological sample is permeabilized to allow analytes to migrate away from the sample and toward the feature array of the substrate. The analytes can interact (e.g., hybridize) with the features (e.g., capture probes therein) on the feature array.

The process 300 can include arranging a feature capture cap relative to the substrate (306). The feature capture cap can be configured to be identical or similar to the feature capture device 104. In some implementations, the feature capture cap is at least partially made of a transparent body and includes a capture surface that is light-activated to be adhesive. The capture surface of the cap can be arranged close to, or abutted to, the feature array on the substrate.

The process 300 can include identifying a region of interest (ROI) on the feature array (308). An ROI is to determine one or more target features that contain analytes of interest on the feature array. In some implementations, an ROI can be identified by a user who can review an image generated for the feature array on the substrate. In alternative implementations, the ROI can be automatically identified on the feature array so that analytes of interest can be obtained by subsequent steps in the process 300.

The process 300 can include emitting light onto the substrate through the cap (310). For example, a beam of light (e.g., a laser beam of suitable wavelength) can be projected through the cap onto the ROI of the feature array on the substrate. When the light hits the capture surface of the cap, the capture surface can absorb the energy and become locally adhesive. Then, the adhesive area of the capture surface can attach target features in the ROI so that the target features in the ROI are removed from the feature array on the substrate.

The process 300 can include removing the cap (312). The cap with the target features attached to the capture surface can be pulled away from the substrate. The process 300 can include removing the target features from the cap (314). Various methods can be used to retrieve the target features from the capture surface of the cap. For example, the target features can be removed from the cap by a dissolvent for dissolving the materials coated on the capture surface.

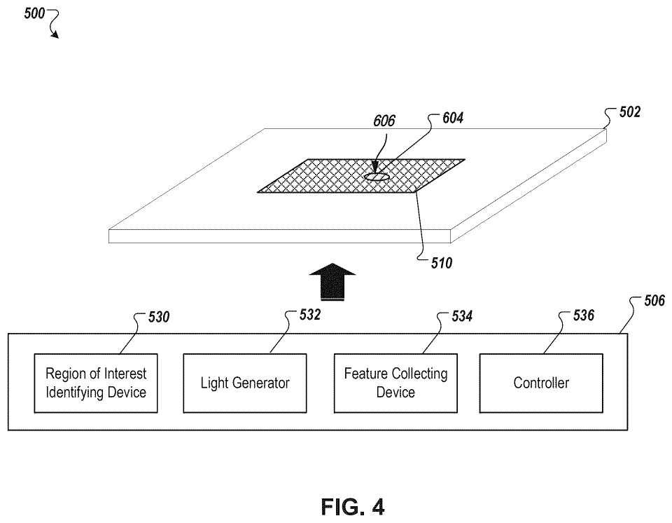

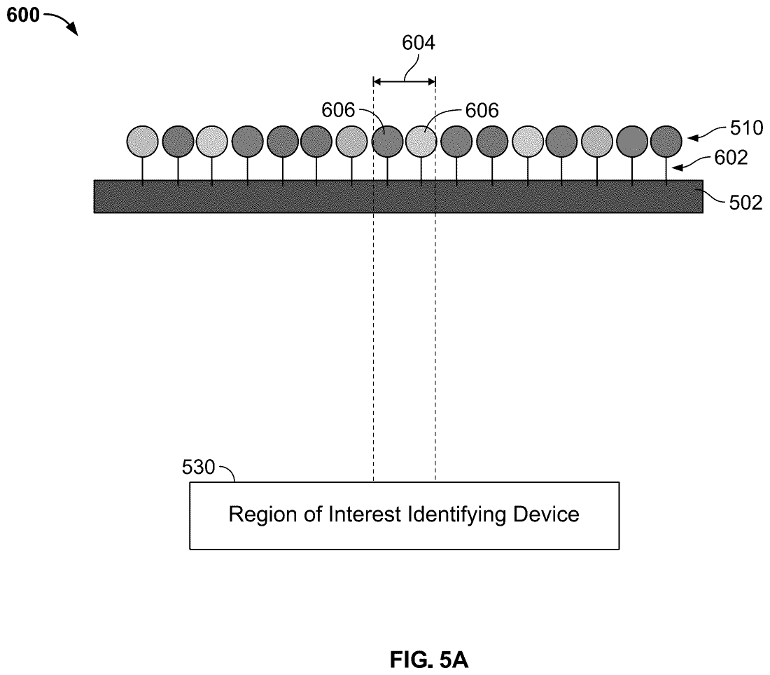

Referring to FIGS. 4-6, another exemplary system for preparing a biological sample is described. In some implementations, the system is configured to obtain one or more analytes (or intermediate agents, such as analyte capture agents) that are spatially targeted. For example, the system can be configured to remove one or more beads that contain analytes of interest from a substrate by projecting light toward the beads of interest on the substrate.

FIG. 4 illustrates an exemplary system 500 for preparing a biological sample. The system 500 includes a substrate 502 and a targeted sample preparation system 506. In some implementation, the substrate 502 and the targeted sample preparation system 506 can be configured similarly to the substrate 102 and the targeted sample preparation system 106 described herein.

In some implementations, the substrate 502 is configured to position an array of features 510. The feature array 510 can include spatially-barcoded capture probes, or an array of beads with such capture probes. The array of features 510 can be directly or indirectly attached or fixed to the substrate 502, or disposed on the substrate 502 in various manners described herein. The array of features 510 can include spatially-barcoded features for array-based spatial analysis, as described herein.

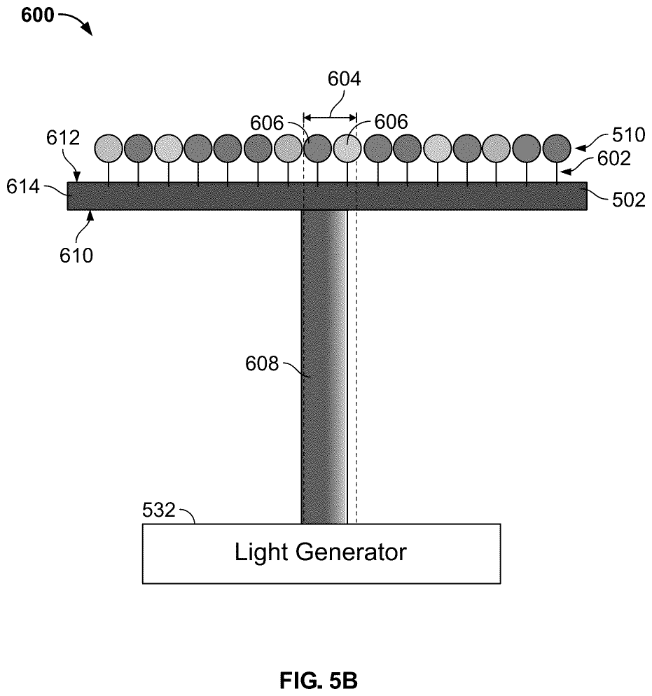

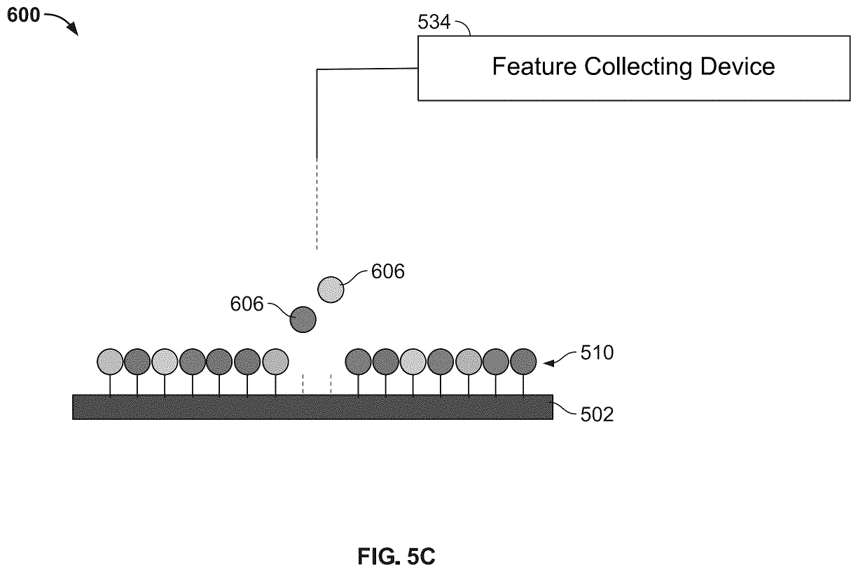

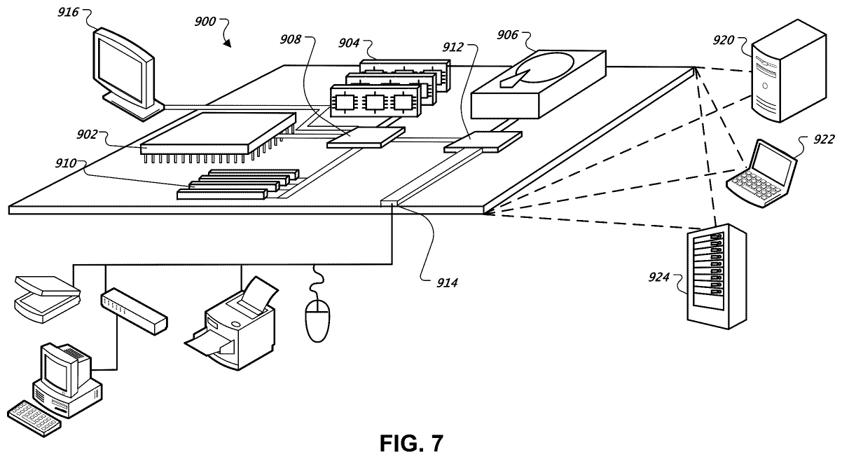

In this example, the feature array 510 can be attached to the substrate 502 with photo-sensitive bonds 602 (FIG. 5A), such as cleavable linkers. An exemplary photo-sensitive bond (e.g., cleavable capture probe) is described herein, for example with reference to FIG. 7. For example, the photo-sensitive bonds 602 can be cleaved when exposed to light, such as UV light. Example linkers of the photo-sensitive bond include -amino-3-(2-nitrophenyl) propionic acid (ANP), phenacyl ester derivatives, 8-quinolinyl benzenesulfonate, dicoumarin, 6-bromo-7-alkixycoumarin-4-ylmethoxycarbonyl, a bimane-based linker, and a bis-arylhydrazone based linker.

The features in the feature array 510 can include analytes captured from a biological sample, such as a cell or a tissue. Various systems and methods can be used to capture analytes from a sample using the feature array 510. For example, the substrate 502 with the feature array 510 can be used for array-based spatial analysis, as described herein. For example, an array-based spatial analysis method described herein can be used to transfer one or more analytes from a sample to the feature array 510 on the substrate. Each feature may be associated with a unique spatial location on the array, and used to identify the analytes and the spatial location of each analyte based on the relative spatial location of the feature to which that the analyte is bound on the feature array 510. Example array-based spatial analysis methods are further described herein.

The targeted sample preparation system 506 is configured to identify a region of interest (ROI) 604 on the feature array 510 and retrieve selected features from the feature array 510 by cleaving the linkers of the features. In some implementations, the targeted sample preparation system 506 can include a ROI identifying device 530, a light generator 532, a feature collecting device 534, and a controller 536.

The ROI identifying device 530 is configured to identify a ROI 604 on the feature array 510 so that one or more features are selectively retrieved from the feature array 510. In some implementations, the ROI identifying device 530 can take an image of the feature array 510 on the substrate 502 in real-time and provide the image to a user so that the user can visualize the feature array 510. For example, the ROI identifying device 530 can include, or be connected to, a display device (e.g., a display screen), and stream the image of the feature array 510 to the display device that can output the image. Examples of the ROI identifying device 530 can include a microscope, a camera, or other suitable imaging devices configured to visualize the feature array 510 of the substrate 502.

In some implementations, the ROI identifying device 530 is configured to automatically identify a ROI on the feature array 510 based on one or more inputs or factors, such as particular types of analytes of interest, particular research purposes, etc. For example, the ROI identifying device 530 can employ a machine learning model that is trained for one or more analysis or research purposes, and configured to automatically determine a ROI on the feature array 510 to meet a particular analysis or research purpose.

In some implementations, a biological sample can be stained to facilitate visualization. Example methods for staining a sample are described herein. The ROI identifying device 530 can capture an image of the stained sample. Optionally, the ROI identifying device 530 can interpret low pass sequencing data (e.g., via amplification of second strand synthesis on the sample removed from the substrate). The removed sample can be placed back on the substrate. The ROI identifying device 530 can visually identify a region of interest on the image based on the stain and/or the sequencing data that have been optionally obtained. The ROI identifying device 530 can be used to permit a user to select a ROI based on the stain image. In some implementations, the ROI identifying device 530, which may employ a machine learning algorithm, can automatically identify and mark a ROI on the stained image. In some implementations, the user can control the light generator 532 to emit light toward the identified ROI on the feature array 510. In other implementations, the light generator 532 can be automatically controlled and guided to be placed so that light is emitted onto the identified ROI of the feature array 510.

The light generator 532 is configured to generate and emit light onto the substrate 502. In some implementations, the light generator 532 is manually controllable to be positioned relative to the substrate 502, and also manually controllable to generate and emit light to a particular area (e.g., the identified ROI) on the feature array 510 of the substrate 502. For example, the light generator 532 includes a user interface (e.g., physical or virtual buttons, switches, or other suitable input devices) for receiving a user input of controlling the light generator 532. Alternatively or in addition, the light generator 532 is configured to automatically adjust its position relative to the substrate 502 and generate and emit light toward a particular area (e.g., the identified ROI) on the feature array 510 of the substrate 502.

The feature collecting device 534 is configured to retrieve target features from the substrate 502. For example, when the linkers are cleaved and the features are released from the substrate 502, the feature collecting device 534 is used to collect the released features. The feature collecting device 534 can use various configurations and methods to retrieve the features removed from the substrate 502. In some implementations, the feature collecting device 534 can include one or more reservoirs that each contain a fluid and is used to collect the removed target features 606 therein. In some implementations, the feature collecting device 534 can include a plurality of reservoirs used to separately collect target features of different characteristics (e.g., physical properties, cell types, gene expression profiles, disease vs. normal, etc.).

The controller 536 can be configured to control the ROI identifying device 530, the light generator 532, and/or the feature collecting device 534. In some implementations, the controller 536 is configured to control the ROI identifying device 530 to identify the ROI on the feature array 510 and receive data indicative of the identified ROI. The controller 536 can control the light generator 532 to arrange or orient the light generator 532 in a desired position relative to the feature array 510 on the substrate 502, and/or generate and project light onto the identified ROI on the feature array 510 of the substrate 502 so that the features on the identified ROI are removed from the substrate 502. The controller 536 can further control the feature collecting device 534 to collect the removed features for further analysis.

FIGS. 5A-C illustrate an exemplary process 600 for performing spatially-targeted feature capture. In this example, the process 600 is illustrated to be performed using the system 500 described in FIG. 4. Referring to FIG. 5A, the process 600 can be performed after the feature array 510 on the substrate 502 has captured analytes from a biological sample. As described herein, the feature array 510 can be attached to the substrate 502 with photo-sensitive bonds 602, such as linkers that are cleavable when exposed to light.

In some implementations, the ROI identifying device 530 can be used to identify a region of interest (ROI) 604 in the feature array 510 on the substrate 502. The ROI 604 is determined to include one or more target features 606 among the feature array 510. As described herein, the ROI identifying device 530 can provide a visual image of the feature array 510 to a user so that the user can identify the ROI 604 from the visual image of the feature array 510. In addition or alternatively, the ROI identifying device 530 can automatically identity the ROI 604, and mark the ROI 604 on the image of the feature array 510, so that light can be accurately projected onto the ROI 604.

Referring to FIG. 5B, the light generator 532 is used to emit light 608 onto the ROI 604 of the feature array 510. In some implementations, the light generator 532 generates a beam of light 608 of suitable wavelength and projects the light beam 608 onto the ROI 604. For example, the light beam 608 can be an ultraviolet (UV) laser. Alternatively, the light generator 532 can include a digital micro-mirror device.

In some implementations, the light 608 is projected onto a bottom surface 610 of the substrate 502 that is opposite to a top surface 612 of the substrate 502 on which the feature array 510 is placed. The light 608 that enters the bottom surface 610 of the substrate 502 passes through a transparent body 614 of the substrate 502 and reaches the top surface 612 of the substrate 502. The light 608 can locally cleave the photo-sensitive bonds 602 in the ROI 604 so that the target features 606 in the ROI 604 are released from the substrate 502.

Referring to FIG. 5C, the target features 606 are released from the substrate 502 when the photo-sensitive bonds 602 are cleaved by the light 608 projected onto the ROI 604. In some implementations, the feature collecting device 534 can be used to collect the target features 606. As described herein, for example, the feature collecting device 534 can include a reservoir that contains a fluid, and the removed target features 606 can be collected in the reservoir. In some implementations, a plurality of reservoirs can be provided and used to separately collect target features 606 of different characteristics (e.g., physical properties, cell types, gene expression profiles, disease vs. normal, etc.). For example, a first reservoir can be used to collect target features 606 having a first characteristic while a second reservoir can be used to collect target features 606 having a second, different characteristic.

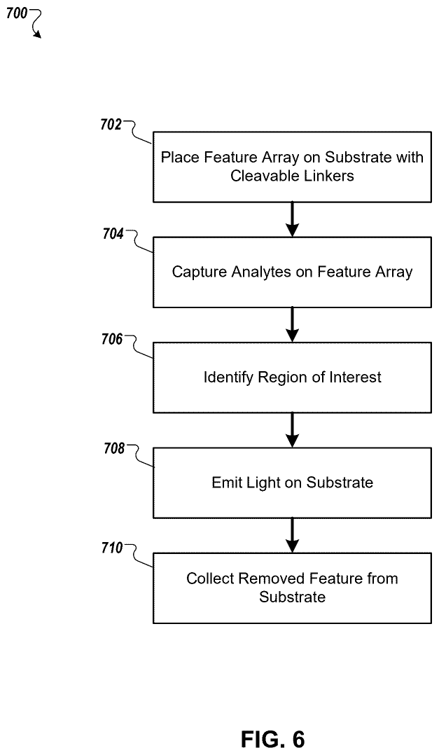

FIG. 6 is a flowchart of an exemplary process 700 for performing spatially-targeted feature capture. In some implementations, the process 700 can be at least partially performed using the system 100 described in FIG. 4. In addition or alternatively, the process 700 can be used to implement at least part of the process 600 described in FIGS. 5A-5C.

The process 700 can include placing a feature array on a substrate with cleavable linkers (702). The feature array can include spatially-barcoded capture probes, or an array of beads with such capture probes or functionality. Additional description of spatially-barcoded capture probes is provided herein; see also, for example, WO 2020/176788 (e.g., Section (II)(b), such as subsections (i)-(vi))), U.S. Patent Application Publication No. 2020/0277663 (e.g., Section (II)(b) (e.g., subsections (i)-(vi))), and/or U.S. Patent Application Publication No. 2019/0264268 (e.g., paragraphs [0088]-[0109]). For example, the feature array can be directly or indirectly attached or fixed to the substrate. Other methods can be used to place the feature array on the substrate. The feature array can be attached to the substrate using cleavable linkers or other types of photo-sensitive bonds as described herein. The linkers are cleavable when exposed to light of certain characteristics (e.g., wavelength). For example, the linkers can be selected to be cleaved when exposed to a UV light.

The process 700 can include capturing analytes from a biological sample on the feature array of the substrate (704). Various methods can be used for capturing analytes from a sample. In one example, a biological sample is permeabilized to allow analytes to migrate away from a sample and toward the feature array of the substrate. The analytes can interact with the features (e.g., capture probes therein) on the feature array.

The process 700 can include identifying a region of interest (ROI) on the feature array (706). An ROI is used to determine one or more target features that contain analytes of interest on the feature array. In some implementations, an ROI can be identified by a user who can review an image generated for the feature array on the substrate. In alternative implementations, the ROI can be automatically identified on the feature array so that the analytes of interest can be obtained by subsequent steps in the process 700.

The process 700 can include emitting light onto the substrate through the cap (708). For example, a beam of light (e.g., a laser beam of suitable wavelength) can be projected onto the ROI of the feature array of the substrate. When the light hits the cleavable linkers in the ROI, the linkers are cleaved so that the features attached to the linkers are released from the substrate.

The process 700 can include collecting the removed target features (710). Various methods can be used to retrieve the target features from the capture surface of the cap. For example, the target features removed from the substrate can be collected within a fluid of a particular type contained in a reservoir.

FIG. 7 is a block diagram of computing devices 900 that may be used to implement the systems and methods described in this document, as either a client or as a server or plurality of servers. For example, at least some of the components described in FIG. 7 can be used to implement the targeted sample preparation system 106, 506 and other instruments, apparatuses, and devices described herein. Computing device 900 is intended to represent various forms of digital computers, such as laptops, desktops, workstations, personal digital assistants, servers, blade servers, mainframes, and other appropriate computers. The components shown here, their connections and relationships, and their functions, are meant to be examples only, and are not meant to limit implementations described and/or claimed in this document.

Computing device 900 includes a processor 902, memory 904, a storage device 906, a high-speed interface 908 connecting to memory 904 and high-speed expansion ports 910, and a low speed interface 912 connecting to low speed bus 914 and storage device 906. Each of the components 902, 904, 906, 908, 910, and 912, are interconnected using various busses, and may be mounted on a common motherboard or in other manners as appropriate. The processor 902 can process instructions for execution within the computing device 900, including instructions stored in the memory 904 or on the storage device 906 to display graphical information for a GUI on an external input/output device, such as display 916 coupled to high-speed interface 908. In other implementations, multiple processors and/or multiple buses may be used, as appropriate, along with multiple memories and types of memory. Also, multiple computing devices 900 may be connected, with each device providing portions of the necessary operations (e.g., as a server bank, a group of blade servers, or a multi-processor system).

The memory 904 stores information within the computing device 900. In one implementation, the memory 904 is a volatile memory unit or units. In another implementation, the memory 904 is a non-volatile memory unit or units. The memory 904 may also be another form of computer-readable medium, such as a magnetic or optical disk.

The storage device 906 is capable of providing mass storage for the computing device 900. In one implementation, the storage device 906 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer-or machine-readable medium, such as the memory 904, the storage device 906, or memory on processor 902.

The high-speed controller 908 manages bandwidth-intensive operations for the computing device 900, while the low speed controller 912 manages lower bandwidth-intensive operations. Such allocation of functions is an example only. In one implementation, the high-speed controller 908 is coupled to memory 904, display 916 (e.g., through a graphics processor or accelerator), and to high-speed expansion ports 910, which may accept various expansion cards (not shown). In the implementation, low-speed controller 912 is coupled to storage device 906 and low-speed expansion port 914. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 900 may be implemented in a number of different forms, as shown in the figure. For example, it may be implemented as a standard server 920, or multiple times in a group of such servers. It may also be implemented as part of a rack server system 924. In addition, it may be implemented in a personal computer such as a laptop computer 922. Alternatively, components from computing device 900 may be combined with other components in a mobile device. Each of such devices may contain one or more of computing device 900, and an entire system may be made up of multiple computing devices 900 communicating with each other.

Additionally computing device 900 can include Universal Serial Bus (USB) flash drives. The USB flash drives may store operating systems and other applications. The USB flash drives can include input/output components, such as a wireless transmitter or USB connector that may be inserted into a USB port of another computing device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434): 1463-1467, 2019; Lee et al., Nat. Protoc. 10 (3): 442-458, 2015; Trejo et al., PLOS ONE 14 (2): e0212031, 2019; Chen et al., Science 348(6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d) ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., proteins) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via next-generation sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up-and/or down-regulated genes and/or proteins in diseased tissue; characterization of tumor microenvironments (e.g., differentiate immune cells from cancer cells); characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21;45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly (A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

1. A system comprising:
a substrate comprising a feature array thereon, wherein the feature array comprises a plurality of features and analytes, intermediate agents, or a combination thereof that were previously captured by the plurality of features from a biological sample;
a feature capture block including a capture surface, the capture surface configured to face the feature array on the substrate;
a light generator configured to emit light; and
a controller configured to activate the light generator to emit the light toward a portion of the capture surface of the feature capture block that corresponds to a region of interest on the feature array to expose the portion of the capture surface to the light such that a light-activated adhesive on the portion of the capture surface is activated to permit the capture surface to capture at least one feature from the feature array, the at least one feature comprising at least one element of the analytes, the intermediate agents, or the combination thereof.

2. The system of claim 1, wherein the feature capture block includes a transparent body to permit the light to pass through the transparent body,
wherein the transparent body of the feature capture block has a light entering surface onto which the light is emitted, the light passing through the transparent body and directed toward the capture surface, and
wherein the light entering surface is arranged to be opposite to the capture surface.

3. (canceled)

4. The system of claim 1, wherein the feature capture block is arranged such that the capture surface contacts the feature array on the substrate as the capture surface captures the at least one feature from the feature array.

5. The system of claim 1, wherein the feature capture block is arranged such that the capture surface is spaced apart from the feature array on the substrate as the capture surface captures the at least one feature from the feature array.

6. The system of claim 1, wherein the light comprises a laser having a predetermined wavelength, the laser comprising an ultraviolet (UV) laser.

7. The system of claim 1, further comprising:
a microscope configured to visualize the region of interest on the feature array.

8. The system of claim 1, wherein the feature array comprises a spatially-barcoded bead array having a plurality of beads being uniform in diameter and arranged in a monolayer, and wherein the plurality of beads corresponds to the plurality of features.

9. A system comprising:
a substrate comprising photo-sensitive bonds and a feature array attached to the substrate via the photo-sensitive bonds, wherein the feature array comprises a plurality of features and analytes, intermediate agents, or a combination thereof that were previously captured by the plurality of features from a biological sample;
a light generator configured to emit light;
a controller configured to activate the light generator to emit the light toward a portion a of the feature array to cleave the photo-sensitive bonds in a region of interest and permit for at least one feature within the region of interest of the feature array to be released from the substrate, the at least one feature comprising at least one element of the analytes, the intermediate agents, or the combination thereof; and
a microscope configured to visualize the region of interest on the feature array.

10. The system of claim 9, wherein the substrate includes a transparent body having a first surface and a second surface, wherein the first surface is configured to place the photo-sensitive bonds and the feature array, the first surface being arranged to be opposite to the second surface, and
wherein the light generator is configured to emit the light onto the second surface of the substrate such that the light passes through the transparent body and reaches the photo-sensitive bonds on the first surface of the substrate.

11. The system of claim 9, further comprising:
a feature collecting device comprising a reservoir into which the at least one feature released from the feature array is received.

12. The system of claim 9, wherein the photo-sensitive bonds include photo-cleavable linkers, and
wherein the photo-cleavable linkers include at least one photo-sensitive chemical bond selected from 3-amino-3-(2-nitrophenyl) propionic acid (ANP), phenacyl ester derivatives, 8-quinolinyl benzenesulfonate, dicoumarin, 6-bromo-7-alkixycoumarin-4-ylmethoxycarbonyl, a bimane-based linker, and a bis-arylhydrazone based linker.

13. The system of claim 9, wherein the feature array comprises a spatially-barcoded bead array having a plurality of beads being uniform in diameter and arranged in a monolayer, and wherein the plurality of beads corresponds to the plurality of features.

14. The system of claim 9, wherein the light comprises a laser of a predetermined wavelength, the laser comprising an ultraviolet (UV) laser.

15. The system of claim 9, wherein the light generator comprises a digital micro-mirror device.

16. The system of claim 9, further comprising:
a feature collecting device comprising a plurality of reservoirs into which the at least one feature released from the feature array is received.

17. The system of claim 1, further comprising:
a feature retrieving device configured to retrieve the at least one feature from the capture surface.

18. The system of claim 17, wherein the feature retrieving device comprises a chamber that contains a solvent to release the at least one feature from the capture surface.

19. The system of claim 17, wherein the feature retrieving device is configured to discharge a solvent toward the light-activated adhesive on the capture surface to release the at least one feature from the capture surface.

20. The system of claim 1, wherein the controller is configured to identify the region of interest by determining that the one or more features of the plurality of features of the feature array contains the at least one element of the analytes, the intermediate agents, or the combination thereof.

21. The system of claim 1, wherein the controller is configured to identify the region of interest on the feature array by:
providing an image of the feature array to a user; and
receiving a selection by the user indicative of the region of interest.
